(19) United States
(12) Patent Application Publication
(10) Pub. No.: US 2006/0257438 A1
(43) Pub. Date: Nov. 16, 2006

(54) Polyalkylene materials

(75) Inventors: Marcel P. Breton, Mississauga (CA); San-Ming Yang, Mississauga (CA); Raymond W. Wong, Mississauga (CA)

Correspondence Address: Richard M. Klein, Esq.;FAY, SHARPE, FAGAN, MINNICH & McKEE, LLP, SEVENTH FLOOR, 1100 SUPERIOR AVENUE, CLEVELAND, OH, 44114-2579, US

(73) Assignee: XEROX CORPORATION

(21) Appl. No.: 11/352,817

(22) Filed: Feb. 13, 2006

Related U.S. Application Data
(63) Continuation-in-part of application No. 11/126,745, filed on May 11, 2005.

(51) Int. Cl. A61K 8/81

(52) U.S. Cl. 424/401

(57) ABSTRACT

The disclosure provides, in various embodiments, a method for fractionating a polyalkylene, and the fractionated polyalkylene produced thereby. The method includes, for example, separating, from a starting polyalkylene, a first portion of a polyalkylene having a Mw less than the Mw of the starting polyalkylene. Also included are bichromal balls or beads comprising the fractionated polyalkylene, such as the first portion of a polyalkylene.

### PRIORITY

The present application is a continuation-in-part of and claims priority under 35 U.S.C. § 120 to U.S. Ser. No. 11/126,745, filed May 11, 2005, the entire disclosure of which is incorporated herein by reference.

### CROSS-REFERENCE TO RELATED APPLICATIONS

Attention is directed to commonly-assigned, currently pending Attorney Docket No. 20041681-US-NP, U.S. patent application Ser. No. ______, filed ______, entitled “Polyalkylene Materials”; patent application Ser. No. 11/273,789, filed Nov. 14, 2005, entitled “Crystalline Wax”; U.S. patent application Ser. No. 11/273,895, filed Nov. 14, 2005, entitled “Crystalline Wax”; U.S. patent application Ser. No. 11/273,748, filed Nov. 14, 2005, entitled “Toner Having Crystalline Wax”; U.S. patent application Ser. No. 11/273,751, filed Nov. 14, 2005, entitled “Toner Having Crystalline Wax”; and U.S. patent application Ser. No. 11/274,459, filed Nov. 14, 2005, entitled “Toner Having Crystalline Wax”. The disclosures of these patent applications are hereby incorporated by reference in their entirety.

### BACKGROUND

The present disclosure is generally directed, in various exemplary embodiments, to methods of separating or fractioning polyalkylene materials, such as polyalkylene waxes. The present disclosure also relates to products produced utilizing the separated or fractionated materials, such as microencapsulated Gyricon or bichromal beads.

High molecular weight (Mw) waxes are used in Gyricon devices, which are utilized in electronic signage. It is found that the contrast ratio of Gyricon devices can be increased if fractionated polyalkylene waxes are used in these devices.

In this regard, bichromal balls, or Gyricon beads as sometimes referred to in the art, are tiny spherical balls, such as micron-sized wax beads, which have an optical and an electrical anisotropy. These characteristics generally result from each hemisphere surface or side having a different color, such as black on one side and white on the other, and electrical charge, i.e., positive or negative. Depending on the electrical field produced, the orientation of these beads will change, showing a different color (such as black or white) and collectively create a visual image.

For example, reusable signage or displays can be produced by incorporating the tiny bichromal beads in a substrate such as sandwiched between thin sheets of a flexible elastomer and suspended in an emulsion. The beads reside in their own cavities within the flexible sheets of material. Under the influence of a voltage applied to the surface, the beads will rotate to present one side or the other to the viewer to create an image. The image stays in place until a new voltage pattern is applied using software, which erases the previous image and generates a new one. This results in a reusable signage or display that is electronically writable and erasable.

Numerous patents describe bichromal balls or beads, their manufacture, incorporation in display systems or substrates, and related uses and applications. Exemplary patents include, but are not limited to: U.S. Pat. Nos. 5,262,098; 5,344,594; 5,604,027 reissued as U.S. Pat. No. Re 37,085; 5,708,525; 5,717,514; 5,739,801; 5,754,332; 5,815,306; 5,900,192; 5,976,428; 6,054,071; 5,989,629; 6,235,395; 6,419,982; 6,235,395; 6,419,982; 6,445,490; and 6,703,074; all of which are hereby incorporated by reference.

However, some polyalkylene waxes fail to meet one or more of the desired requirements for bichromal balls or beads. In this regard, waxes may exhibit large batch-to-batch variations, high polydispersity indexes (PDI), skewnesses in Mw distribution, etc. These undesired material characteristics create inconsistent results.

Some commercially available polyalkylene waxes, such as POLYWAX™ 655 and 500 (Baker-Petrolite Corp.), have wide Mw distributions with carbon chain lengths ranging from about 30 to about 70 carbons (POLYWAX 500) and from about 30 to about 100 carbons (POLYWAX 655). Additionally, these waxes have a high content of low Mw fractions (fractions comprising carbon chain lengths of from about 50 carbons or less). The low Mw fraction in POLYWAX 500 is around 50% by weight, and the low Mw fraction in POLYWAX 655 is around 40% by weight. Low Mw materials lower the onset of wax melting (lower the offset temperature) and also weaken the mechanical strength of the solidified waxes.

Moreover, there are only a limited number of large scale methods available to purify wax material. Distillation is one method typically used to provide fractionated versions of the waxes. For example, some suppliers of the polyalkylene waxes perform distillation processes on the waxes to supply fractionated or semi-fractionated versions and to achieve narrower Mw distributions. These distillation procedures, however, have drawbacks in that they are expensive and generally limited to lower molecular weight materials.

This disclosure is directed to overcoming one or more of the aforementioned problems and/or others.

### BRIEF DESCRIPTION

In one exemplary embodiment, a method of fractioning a polyalkylene, such as a polyalkylene wax, is provided. The method comprises providing an initial polyalkylene having a weight average molecular weight Mw; combining the polyalkylene with a fluid, wherein the fluid is selected from a group consisting of a) acyclic branched or unbranched hydrocarbons having the general formula CₙH₂ₙ₊₂, in which n is the number of atoms and is, for example, from about 1 to about 30, including from about 5 to about 26, and from about 5 to about 16; and, b) supercritical fluids (“SCF”); and wherein a first portion of the polyalkylene with a weight average molecular weight Mw₁<Mw becomes dissolved in the fluid; separating from the fluid a second portion of the polyalkylene with a weight average molecular weight Mw₂>Mw that is insoluble in the fluid; and, recovering the first portion or the second portion of the polyalkylene material from the fluid. Also disclosed is the fractionated polyalkylene produced by this process.

In another exemplary embodiment, a bichromal ball or bead comprising the fractionated polyalkylene from the above method is provided. These products have several uses including, but not limited to, reusable signage or display applications.

In still another exemplary embodiment, a polyalkylene wax fraction is provided. The polyalkylene wax fraction is obtained by combining an initial polyalkylene having a weight average molecular weight Mw with a fluid, wherein the fluid is selected from the group consisting of a) acyclic branched or unbranched hydrocarbons having the general formula CₙH₂ₙ₊₂, in which n is the number of atoms and is, for example, from about 1 to about 30, including from about 4 to about 26, and from about 5 to about 16; and, b) supercritical fluids, dissolving a first portion of the polyalkylene in the fluid, the first portion having a weight average molecular weight Mw₁ that is from about 0.55 Mw to about 0.95 Mw, separating a second portion of the polyalkylene, the second portion being insoluble in the fluid and having a weight average molecular weight Mw₂ that is from about 1.05 Mw to about 1.45 Mw, and recovering the first potion, wherein the polyalkylene wax fraction comprises the first portion of the polyalkylene wax.

In still another exemplary embodiment, a bichromal ball or bead is provided comprising a colorant; and a polyalkylene wax fraction obtained by extracting the polyalkylene wax fraction from an initial or starting polyalkylene with a fluid; wherein the fluid is selected from the group consisting of hydrocarbons of the formula CₙH₂ₙ₊₂ in which n is the number of atoms and is, for example, from about 1 to about 30, including from about 4 to about 26, and from about 5 to about 16 and supercritical fluids, and recovering the polyalkylene wax fraction, the polyalkylene wax fraction having a polydispersity equal to or less than about 1.30, including less than about 1.07, such as 1.05.

In yet another exemplary embodiment, a bichromal ball or bead is provided comprising a colorant and a carrier comprising a first polyalkylene wax portion separated from a starting polyalkylene wax having a polydispersity index PDI, the polyalkylene wax portion being separated from the starting polyalkylene wax by (i) combining the starting polyalkylene wax with a fluid comprising acyclic branched or unbranched hydrocarbons having the general formula CₙH₂ₙ₊₂, in which n is the number of atoms and is, for example, from about 1 to about 30, including from about 4 to about 26, and from about 5 to about 16, (ii) dissolving the first polyalkylene wax portion in the fluid, and (iii) recovering the first polyalkylene wax portion from the fluid, wherein the first polyalkylene wax portion has a polydispersity index PDI₁, and PDI₁ is less than PDI.

In still a further exemplary embodiment, a fractionated polyalkylene wax is provided, the fractionated polyalkylene wax being obtained by (i) combining a polyalkylene wax having a weight average molecular weight (Mw) and a polydispersity index PDI with a fluid, wherein the fluid is selected from the group consisting of a) acyclic branched or unbranched hydrocarbons having the general formula CₙH₂ₙ₊₂, in which n is the number of atoms and is from about 5 to about 16; and, b) supercritical fluids, (ii) dissolving a first portion of the polyalkylene in the fluid, the first portion having a weight average molecular weight Mw₁ that is less than Mw and a polydispersity index PDI₁ that is less than PDI, (iii) separating a second portion of the polyalkylene wax, the second portion being insoluble in the fluid and having a weight average molecular weight Mw₂ that is greater than Mw, and a PDI₂ that is greater than PDI, and (iv) recovering the first portion, wherein the fractionated polyalkylene wax comprises the first portion of the polyalkylene.

In a further exemplary embodiment, a microencapsulated Gyricon or bichromal bead comprising the separated or fractionated polyalkylene wax from the above method is provided.

These and other non-limiting features of the exemplary embodiments will be more particularly described with regard to the drawings and detailed description set forth below.

### BRIEF DESCRIPTION OF THE DRAWINGS

The following is a brief description of the drawings, which are presented for the purposes of illustrating one or more of the exemplary embodiments disclosed herein and not for the purposes of limiting the same.

FIG. 1 shows the DSC Analysis of separated polyalkylene samples according to one embodiment of the present disclosure;

FIG. 2 shows the HT-GPC statistical analysis of several separated and unseparated polyalkylene samples according to one embodiment of the present disclosure;

FIG. 3 shows a DSC Analysis of a first portion extracted from a polyalkylene wax according to one embodiment of the present disclosure; and

FIG. 4 shows a DSC Analysis for POLYWAX 655, a commercial wax available from Baker-Petrolite Corp.

### DETAILED DESCRIPTION

The disclosure provides, in various embodiments, a method for separating or fractionating a polyalkylene material, and the separated or fractionated polyalkylene material(s) produced thereby. The method generally includes separating low molecular weight polyalkylene fractions from high molecular weight fractions from a starting polyalkylene comprising mixtures of such fractions. For example, a method in accordance with the disclosure includes separating and obtaining, from a starting polyalkylene, a first portion of a polyalkylene having at least one of (i) a Mw less than the Mw of the starting polyalkylene and/or (ii) a polydispersity index less than the polydispersity index of the starting polyalkylene. Also included are microencapsulated Gyricon or bichromal beads comprising a portion separated from the starting polyalkylene, such as the first portion of a polyalkylene.

Solvent extraction techniques may be employed in the present separation method of polyalkylene fractions from a wax. “Solvent extraction” in the embodiments includes, for example, the process of transferring a substance from any matrix to an appropriate liquid phase. For example, a starting polyalkylene with a weight average molecular weight Mw (also referred to herein as “the polyalkylene with Mw”) in the method may serve as the “any matrix” or “solid phase”; and a hydrocarbon, for example, may serve as the appropriate liquid phase. In the separation process, the first portion of the polyalkylene may be substantially transferred or extracted into the fluid, such as, for example, acyclic branched or unbranched hydrocarbons having the general formula CₙH₂ₙ₊₂, in which n is the number of atoms and is, for example, from about 1 to about 30, including from about 4 to about 26, and from about 5 to about 16, while the second portion of the polyalkylene containing high molecular weight fractions can not be substantially extracted or dissolved into the fluid. Sometimes, various leaching techniques may also be employed in the present method.

When a “range” or “group” is mentioned with respect to a particular characteristic of the present disclosure, for example, molecular weight, chemical species, and temperature, it relates to and explicitly incorporates herein each and every specific member and combination of sub-ranges or sub-groups therein whatsoever. Thus, any specified range or group is to be understood as a shorthand way of referring to each and every member of a range or group individually as well as each and every possible sub-ranges or sub-groups encompassed therein; and similarly with respect to any sub-ranges or sub-groups therein.

A starting or initial polyalkylene refers, for example, to a composition comprising hydrocarbon chains such as a polyalkylene. The starting polyalkylene generally is a polyalkylene material such as, for example, a polyalkylene with a weight average molecular weight Mw that has not been subjected to a separation or extraction process in accordance with the present disclosure. A starting polyalkylene may also be referred to herein as a starting polyalkylene wax. Examples of starting polyalkylene waxes include, but are not limited to, polyethylene wax, polypropylene wax, mixture thereof, and any form of ethylene-propylene copolymer wax. In several embodiments of the disclosure, the polyalkylene wax comprises polyethylene wax.

The “polyethylene” used in the disclosure should not be limited to a polymer prepared from ethylene. Polyethylene (PE) waxes may be made from ethylene produced from natural gas or by cracking petroleum naphtha. Ethylene may then be polymerized to produce waxes with various melt points, hardnesses and densities, etc. A polyethylene wax may comprise branched polyethylene, linear polyethylene, or mixture thereof. In typical embodiments, the polyethylene with Mw comprises linear polyethylene.

Commercially available polyalkylene waxes suitable as the starting material include, but are not limited to, polyethylene waxes and functionalized polyethylene waxes such as, for example, those sold under the trade names POLYWAX™ from Baker-Petrolite Corp., AC™ PE wax from Honeywell, LICOWAX™ PE family from Clariant, Synthetic wax from Salsowax, and LUWAX™ from BASF. Some specific examples of suitable wax materials include, but are not limited to, POLYWAX 850, POLYWAX 1000, and POLYWAX 2000.

A starting polyalkylene material is a composition comprising hydrocarbon chains such as polyalkylenes. Other suitable starting polyalkylene include UNILIN™ waxes available from Baker-Petrolite. UNILIN waxes are polyhydroxy compounds that have a broad molecular weight, i.e. from about 300 to about 1500 or more. They are described on Baker-Petrolite's website as long chain primary alcohols composed of approximately 80% primary alcohol and 20% hydrocarbon. An exemplary UNILIN wax is UNILIN 700.

The starting polyalkylene utilized herein has a weight average molecular weight Mw. The value of Mw is not particularly limited. In various embodiment, the value of Mw may broadly range from about 400 to about 15,000. In one embodiment, the starting polyalkylene has a Mw of from about 425 to about 3,000.

Additionally, the starting polyalkylene exhibits a polydispersity index (PDI), which refers to the ratio Mw/Mn, in which Mn is the number average molecular weight of the polymer and Mw is the weight average molecular weight of the polymer. In various embodiments, the PDI of the starting polyalkylene, such as, for example, a polyethylene, with Mw may generally range from that approaching 1 to about 3.0, including from that approaching 1 to about 2.0, and from that approaching 1 to about 1.3.

According to the disclosure, the polyalkylene with Mw may be separated into at least two portions. The first portion polyalkylene has a weight average molecular, Mw₁, and is sometimes referred to as “the first portion polyalkylene with Mw₁”; the second portion polyalkylene has a weight average molecular, Mw₂, and is sometimes referred to herein as “the second portion polyalkylene with Mw₂”. In typical embodiments, separation of the first portion polyalkylene and the second portion polyalkylene is accomplished based on their solubility difference in a fluid, such as, for example, a hydrocarbon fluid as described herein.

The first portion of a polyalkylene generally comprises low Mw fractions comprising hydrocarbon chains of about 50 carbons and less. The first portion of a polyalkylene also generally exhibits a narrow Mw distribution as indicated by its PDI relative to the starting polyalkylene from which the first portion is separated or fractioned.

In some embodiments, the first portion, comprising a low content of a low Mw fraction (i.e., fractions comprising hydrocarbon chains of about 50 carbons and less) is less than about 30% by volume of the starting polyalkylene. In another embodiment, the low Mw portion of a polyalkylene is less than about 10% by volume of the starting polyalkylene. In still yet another embodiment, the low Mw portion that is less than about 5% by volume of the starting polyalkylene.

The Mw₁ value of the first portion is generally less than Mw of the starting polyalkylene. In various embodiments, the Mw₁ value of the first portion polyalkylene relative to the starting polyalkylene Mw may generally range from about 0.55 Mw to about 0.95 Mw. In one embodiment, Mw₁ is in the range of from about 0.70 Mw to about 0.75 Mw. In a specific embodiment, Mw₁≈0.73 Mw, such as, for example, where Mw≈2,746 and Mw₁≈1,999.

The second portion contains the larger hydrocarbon chains from the starting polyalkylene. The Mw₂ value of the second portion is generally greater than Mw of the starting polyalkylene. In various embodiments, the Mw₂ value of the second portion polyethylene relative to the starting polyalkylene may generally range from about 1.05 Mw to about 1.45 Mw. In one embodiment, Mw₂ is in the range of from about 1.20 Mw to about 1.30 Mw. In a specific embodiment, Mw₂≈1.24 Mw, such as, for example, where Mw≈2,746 and Mw₂≈3,418.

The first and second polyalkylene portions may exhibit a polydispersity index (PDI₁ and PDI₂, respectively) that is lower than, equal or about equal to, or greater than PDI of the starting polyalkylene. In one embodiment, the first portion polyalkylene, such as for example a first portion polyalkylene with Mw₁, has a polydispersity index PDI₁ that is less than PDI (i.e. PDI₁<PDI); and the second portion polyalkylene, such as for example, a second portion polyalkylene, with Mw₂ has a polydispersity index PDI₂ which is also less than PDI (i.e. PDI₂<PDI). In various embodiments, both PDI₁ and PDI₂ are in the range of from about 0.78PDI to about 1.05PDI. In one embodiment PDI, is from about 0.90PDI to about 1.0PDI. In another embodiment, PDI, is about 1.30 or less. And in still another embodiment, PDI₁ is about 1.04. In one specific embodiment, PDI≈1.45, PDI₁≈1.28, and PDI₂≈1.27.

Additionally, in some embodiments, a first portion exhibits a relatively narrow melting characteristic as compared to the starting polyalkylene. As used herein, “melting characteristic” refers to the temperature range over which the melting process occurs for a polyalkylene including, but not limited to a starting polyalkylene wax, a first portion of a polyalkylene, or a second portion of polyalkylene. The melting characteristic of a wax may be analyzed by a DSC trace, as is known in the art. Generally, the melting process for a polyalkylene begins or initiates at a first temperature (T₁) and ends at a second temperature (T₂). The peak temperature, as evidenced by a DSC trace, is referred to as the melting point. The melting characteristic is the temperature difference between T₂ and T₁ (i.e., T₂−T₁). In one embodiment, the melting characteristic for a first portion of a polyalkylene is about 50° C. or less. In another embodiment, the melting characteristic for a first portion of a polyalkylene is about 40° C. or less. In still another embodiment, the melting characteristic of a first portion of a polyalkylene is about 40° C.

Additionally, in some embodiments, a first portion exhibits a relatively narrow crystallization characteristic relative to that of the starting polyalkylene. As used herein, “crystallization characteristic” refers to the temperature range over which the crystallization process occurs for a polyalkylene including, but not limited to, a starting polyalkylene wax, a first portion of a polyalkylene, or a second portion of polyalkylene. The crystallization characteristic of a wax may be analyzed by a DSC trace, as is known in the art. Generally, for a polyalkylene, the crystallization process begins or initiates at a first temperature (T₃) and ends at a second temperature (T₄) wherein T₄ is smaller than T₃. The peak temperature, as evidenced by a DSC trace, is referred to as the crystallization point. The crystallization characteristic is the temperature difference between T₃ and T₄ (i.e., T₃−T₄). In one embodiment, the crystallization characteristic for a first portion of a polyalkylene is about 50° C. or less. In another embodiment, the crystallization characteristic for a first portion of a polyalkylene is about 40° C. or less. In still another embodiment, the crystallization characteristic of a first portion of a polyalkylene is about 30° C. or less. Yet, in another embodiment, the second temperature T₄ of the fractionated fraction is from about 1° C. to about 20° C. above the temperature T₄ of the starting polyalkylene. In some embodiments, the fractionated portion of the polyalkylene has a preferred crystallization temperature T₄ that is above 55° C. while the T₄ temperature of the starting polyalkylene is below 50° C.

Generally, the method for fractionalizing and obtaining the first or second portion includes providing a starting polyalkylene material having a weight average molecular weight Mw, combining the starting polyalkylene with a fluid, dissolving a first portion of the polyalkylene with a weight average molecular weight Mw₁<Mw in the fluid, separating a second portion of the polyalkylene with a weight average molecular weight Mw₂ that is insoluble in the fluid, and optionally recovering the first portion. The method may also include washing the second portion with the fluid to dissolve and extract any first portion polyalkylenes that may not have been dissolved or extracted.

The fluid with which the starting polyalkylene is mixed may be selected from (i) a hydrocarbon having the general formula CₙH₂ₙ₊₂ or (ii) a supercritical fluid. In various embodiments, the polyalkylene starting material is mixed with a hydrocarbon fluid selected from acyclic branched or unbranched hydrocarbons having the general formula CₙH₂ₙ₊₂, in which n is the number of atoms and is, for example, from about 1 to about 30, including from about 4 to about 26, and from about 5 to about 16. Such hydrocarbons may comprise a normal (n-) alkane, an isomeric (iso-) alkane, or mixtures thereof. In one embodiment, the hydrocarbon fluid may comprise an isomeric alkane. In another embodiment, the hydrocarbon fluid comprises an isomeric alkane having from about 7 to about 10 carbon atoms.

Exemplary hydrocarbons of the formula CₙH₂ₙ₊₂ may be selected from one or more of the following compounds or mixture thereof:

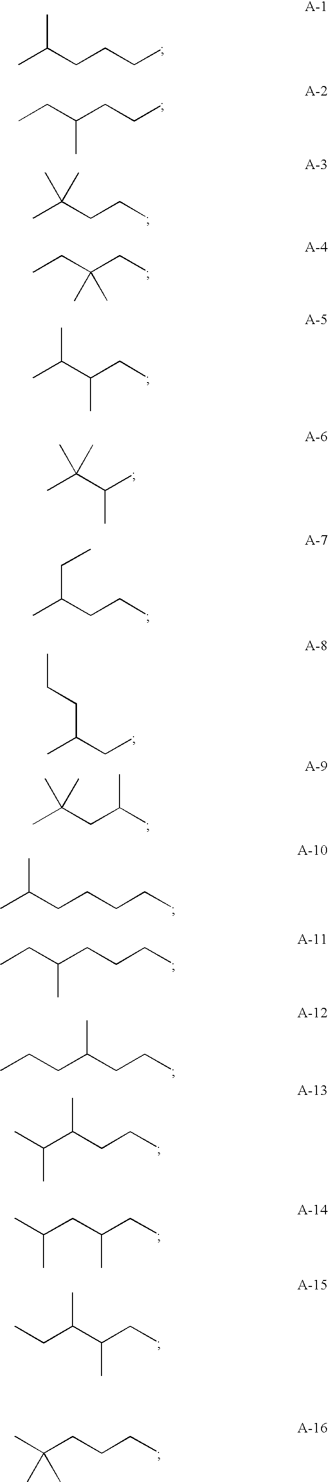

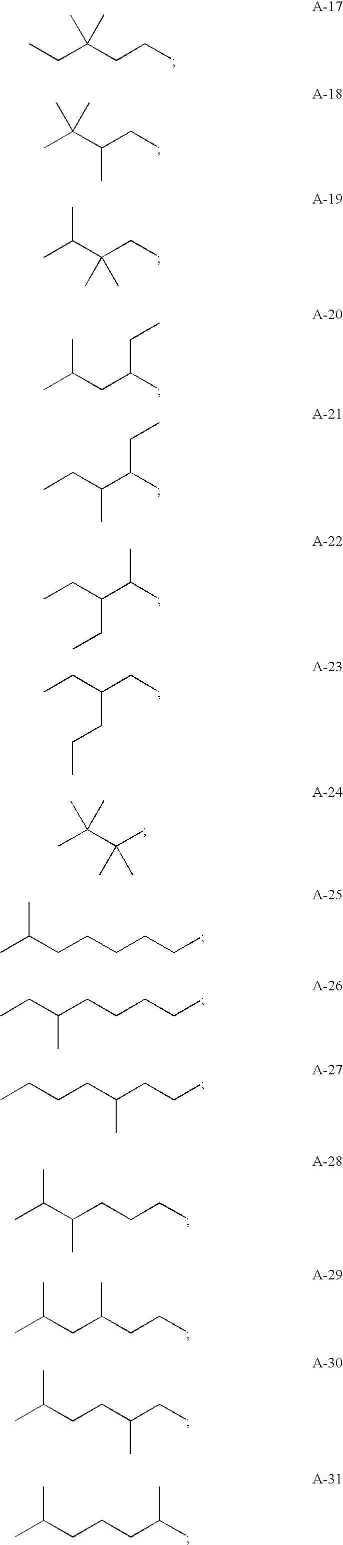

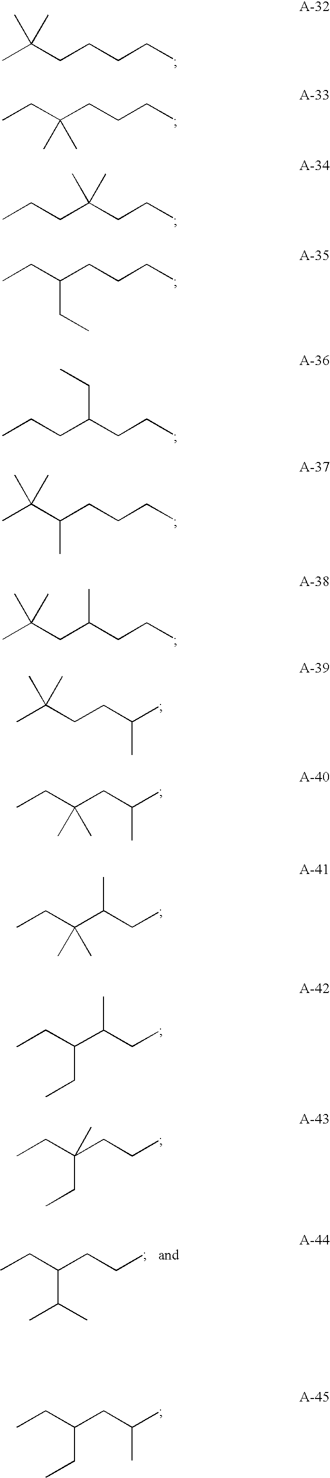

In a specific embodiment, the hydrocarbon fluid comprises the compound having Formula A-9, which is known as 2,2,4-trimethyl pentane [CH₃C(CH₃)₂CH₂CH(CH₃)CH₃] and may be commercially obtained from Exxon-Mobile (Houston, Tex.) under the trade name of ISOPAR™ C.

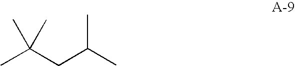

In various embodiments, the weight ratio between the starting polyalkylene, such as, for example, polyethylene, with Mw and the hydrocarbon fluid may generally range from about 1:2 to about 1:8 typically range from about 1:3 to about 1:5. In a specific embodiment, the weight ratio between the starting polyalkylene, such as, for example, polyethylene, with Mw and the hydrocarbon fluid is in the neighborhood of 1:4.

The dissolving process, also referred to herein as extraction, may be accomplished by mixing the starting polyalkylene with a fluid for a sufficient period of time. In one embodiment, the starting polyalkylene may be mixed with a fluid for a period of time of from about 10 minutes to about 10 hours. In another embodiment, the mixing may take place for a period of from about 1 to about 5 hours. In still another embodiment, a starting polyalkylene is mixed with a fluid for about 3 hours to dissolve or extract the first portion.

In several embodiments employing a hydrocarbon fluid as the solvent, the processes may be conducted at an elevated temperature such as above room temperature. In one embodiment the temperature is in the range of from about 45° C. to about 125° C. In another embodiment, the temperature is in the range of from about 65° C. to about 105° C. In still another embodiment, the temperature is from about 60° C. to about 90° C., including about 85° C. In a further embodiment, the temperature is about 70° C. In exemplary embodiments, the method is known as hot solvent extraction. In a specific embodiment, the method is a hot solvent extraction of POLYWAX 2000 (PW2000) by ISOPAR C at 85° C. In another specific embodiment, the method comprises a hot solvent extraction of POLYWAX 1000 (PW1 000) using ISOPAR C at 70° C.

In some embodiments, the first portion of the polyalkylene is recovered from the hydrocarbon fluid solvent. The first portion generally contains low Mw fractions, such as fractions from about 50 carbons or less. A first portion may be recovered by separating the solvent portion, which contains the low Mw fractions, from the second portion of the polyalkylene, such as by decanting, and then cooling the solvent to room temperature to precipitate the first portion of the polyalkylene. The first portion may then be isolated by any suitable method, such as by filtration. Additionally, the second portion may be washed one or more times with the hydrocarbon fluid to further obtain any undissolved or unextracted first portion.

If desired, commonly-known extraction techniques may be used in the method of the disclosure. For example, the method may be conducted with the aid of filter such as vacuum filter, dryer, or combination thereof such as Cogeim filter-dryer; the method may also be conducted with stirring such as 30 RPM; the method may use a sufficiently long operation hour to obtain optimal separation result such as 1-6 hours, for example 3 hours; for a given sample, the method may be repeated as many times as desired, for example, 2-6 times such as 4 times. 4=12 hours; and the raw wax material and the fractionated wax material may be analyzed by DSC and High Temperature GPC (HTGPC).

In other embodiments, a supercritical fluid (“SCF”) can be utilized. A supercritical fluid is disclosed here as any substance at a temperature and pressure above its thermodynamic critical point. It has the unique ability to diffuse through solids like a gas, and dissolve materials like a liquid. Additionally, it can readily change in density upon minor changes in temperature or pressure. These properties make it suitable as a substitute for organic solvents in a process called supercritical fluid extraction. Carbon dioxide and water are the most commonly used supercritical fluids. Examples of other supercritical fluids which can be utilized herein include carbon dioxide by itself or in blends with cosolvents such as methanol, ethanol, propane, ethane, etc.

Supercritical fluids can be regarded as “hybrid solvents” with properties between those of gases and liquids, i.e., a solvent with a low viscosity, high diffusion rates and no surface tension. In the case of supercritical carbon dioxide, the viscosity is in the range of from about 0.02 to about 1.0 cP, where liquids have viscosities of approximately from about 0.5 to about 1.0 cP and gases approximately 0.01 cP, respectively. Diffusivities of solutes in supercritical carbon dioxide are up to a factor 10 higher than in liquid solvents. Additionally, these properties are strongly pressure-dependent in the vicinity of the critical point, making supercritical fluids highly tunable solvents. Of the components shown below, carbon dioxide and water are the most frequently used in a wide range of applications, including extractions, dry cleaning and chemical waste disposal. In polymer systems, ethylene and propylene are also widely used, where they act both as a solvent and as the reacting monomer.

### Critical Properties of Various Solvents

| | | TEMPER- | | |
|---|---|---|---|---|
| | MOLECULAR | ATURE | PRESSURE | DENSITY |
| SOLVENT | WEIGHT | (K) | (BAR) | (G/CM³) |
| Carbon | 44.01 | 304.1 | 73.8 | 0.469 |
| Dioxide | | | | |
| Water | 18.02 | 647.3 | 221.2 | 0.348 |
| Methane | 16.04 | 190.4 | 46.0 | 0.162 |
| Ethane | 30.07 | 305.3 | 48.7 | 0.203 |
| Propane | 44.09 | 369.8 | 42.5 | 0.217 |
| Ethylene | 28.05 | 282.4 | 50.4 | 0.215 |
| Propylene | 42.08 | 364.9 | 46.0 | 0.232 |
| Methanol | 32.04 | 512.6 | 80.9 | 0.272 |
| Ethanol | 46.07 | 513.9 | 61.4 | 0.276 |
| Acetone | 58.08 | 508.1 | 47.0 | 0.278 |

References:
R. C. Reid, J. M. Prausnitz and B. E. Poling, The properties of gase and liquids, 4^{th} ed., McGraw-Hill, New York, 1987.

Suitable supercritical fluid (SCF) fractionation techniques include, but are not limited to the techniques described by Britto et al. in J. of Polymer Science: Part B: Polymer Physics, Vol. 37, 553-560 (1999) and references therein, the disclosure of which is included herein its entirety by reference. In a first method, an isothermal supercritical fluid fractionation uses supercritical fluids to fractionate polymers into narrow molecular weight distribution fractions. In this technique, pressure is used to vary the solvation power of the supercritical solvent, e.g., propane. The higher the pressure, the higher the solvation power of the solvent. In a second method, the supercritical solvent is used to fractionate the polyalkylene based on crystallinity. This technique is called: “Critical Isobaric Temperature Rising Elution Fractionation.”

In typical embodiments, the separation method of this disclosure is scaleable. For example, in a single operation, at least 30 kg, typically at least 40 kg, more typically at least 50 kg of polyalkylene, such as polyethylene, with Mw (e.g., POLYWAX 2000) may be subject to the method.

In exemplary embodiments, the method not only can solve the high temperature Gyricon tolerance problem, but it also alleviates the batch-to-batch variability exhibited in waxes such as, for example, of POLYWAX from Baker-Petrolite Corp. This batch-to-batch variability has a negative effect on final device performance. The root cause is the variability in the distribution of Mw of POLYWAX. After implementation of the present method, narrowing of the Mw distribution is observed, and this eliminates the wax variability. Also, raw wax material has usually a broader melting characteristic. After the purification process of the method, it is shown that the melting point becomes sharper, which can possibly enhance the toner fusing properties and also the jetting conditions.

The disclosure further provides a microencapsulated Gyricon bead comprising a separated/fractionated polyalkylene wax such as, for example, one of the first portion polyalkylene with Mw₁ or the second portion polyalkylene with Mw₂ obtained from the method as illustrated above. Generally, a microencapsulated Gyricon bead includes a bichromal sphere formed of a first material and a second material. A third liquid material such as transparent oil surrounds the bichromal sphere and functions as a rotation medium for the bichromal sphere. The bichromal sphere and the surrounding third material may be disposed within a fourth solid material.

The first material and the second material divide the bichromal sphere into two hemispheres. The hemispheres, namely the first material and the second material, are both optically isotropic and electrically isotropic. In various exemplary embodiments, the first material and the second material are pigmented polymers, with different surface colors between each other.

In various embodiments, the base polymer for one or two hemispheres of the bichromal sphere may comprise the fractionated polyalkylene wax of this disclosure such as fractionated POLYWAX 1000 and/or POLYWAX 2000. For example, a lighter or white pigment may be dispersed into the white/lighter hemisphere. Titanium dioxide white pigment such as is DUPONT™ R104 TiO₂ pigment may be used for this purpose. On the black/color hemisphere of the bichromal sphere, a variety of black pigments may be used, such as manganese ferrite and carbon black, e.g. FERRO™ 6331 manufactured by the Ferro Corporation, Cleveland, Ohio. Of course, other suitable pigments can also be used such as modified carbon blacks, magnetites, ferrites, and color pigments.

The bichromal spheres are relatively small, for example from about 2 to about 200 microns in diameter, and typically from about 30 to about 120 microns in diameter. In media that are active in an electric field, the bichromal spheres have a net dipole due to different levels of charge on the two sides of the sphere. An image is formed by the application of an electric field to the bichromal spheres, which rotates the bichromal spheres to expose one color or the other to the viewing surface of the media. The spheres may also have a net charge, in which case they will translate in the electric field as well as rotate. When the electric field is reduced or eliminated, the spheres ideally do not rotate further; hence, both colors of the image remain intact.

In some embodiments, crystalline materials are ideal for the production of high quality bichromal spheres. This is possibly due to the crystalline material's ability to transition rapidly from a low viscosity liquid to a solid as they cool by moving through the air. Non-separated polyalkylenes, such as the starting polyalkylene wax, have little or no crystalline properties. This is due to the relatively large size range of the molecules, but the fractionated or extracted polyalkylenes typically have stronger crystalline properties. By “crystalline”, it is referred to materials that remain solid as the temperature is increased. Specifically, when the melting point of the material is reached, a crystalline material will melt, sometimes abruptly, and become a low viscosity liquid. This is a desired feature of the crystalline material. For example, this property preserves the hemispherical bichromal quality of the beads after they are formed by the break-up of the Taylor instability jets formed on the edge of the spinning disk during manufacture.

In some embodiments, the fractionated polyalkylene wax, such as the fractionated portion of POLYWAX 2000, is more desired if it has a linear structure and/or has a lower polydispersity such as PDI₁ and PDI₂, which aids in the material having a high crystalline property. Also desired are crystalline materials having a relatively low melting point of from about 50 to about 180° C., and more specifically from about 80 to about 130° C. Further, it is desirable that the crystalline material have a carbon content of from about 18 to about 1,000, including from about 50 to about 200 carbon atoms.

The fabrication of certain bichromal spheres is known, for example, as set forth in U.S. Pat. No. 4,143,103, the disclosure of which is fully incorporated herein by reference, wherein the sphere is comprised of black polyethylene with a light reflective material, for example, indium, sputtered on one hemisphere. Also in U.S. Pat. No. 4,438,160, further included fully herein by reference, a rotary ball is prepared by coating white glass balls of about 50 microns in diameter, with an inorganic coloring layer such as co-deposited MgF₂ and chromium by evaporation. In a similar process, there is disclosed in an article entitled “The Gyricon—A twisting Ball Display”, published in the proceedings of the S.I.D., Vol. 18/3 and 4 (1977), a method for fabricating bichromal balls by first heavily loading chromatic glass balls with a white pigment such as titanium oxide, followed by coating from one direction in a vacuum evaporation chamber with a dense layer of nonconductive black material which coats only one hemisphere. The process set forth in this article is also fully incorporated herein by reference.

Also in U.S. Pat. No. 4,810,431 by Leidner, further fully incorporated herein by reference, there is disclosed a process for generating spherical particles by (a) coextruding a fiber of a semi-circular layer of a polyethylene pigmented white and a black layer of polyethylene containing magnetite, (b) chopping the resultant fiber into fine particles ranging from 10 microns to about 10 millimeters, (c) mixing the particles with clay or anti-agglomeration materials, and (d) heating the mixture with a liquid at about 120° C. to spherodize the particles, followed by cooling to allow for solidification.

In another method, the bichromal beads used in the fabrication of display media such as Gyricon electric paper are formed by wetting the top and bottom surfaces of a spinning disk with two different pigmented molten solids. These streams combine at the edge of the disk and, driven by a Taylor instability, they form a series of jets emanating from the edge of the disk. In particular, a 3 inch diameter disk will have about 300 such jets. Each jet is seen with high speed video to be comprised of two very distinct parts corresponding to the two pigmented liquids used, with no apparent mixing within the jet. The jets subsequently break up into spheres by the Rayleigh instability. Again, with high speed video, it can be seen that close to the jet break-up points, these spheres are very high quality, hemispherical bichromal spheres.

The third material may be any dielectric liquid, such as the ISOPARs by the Exxon Corporation, and 1 or 2 centistoke silicone 200 liquid by the Dow Corning Corporation. The fourth material/skin may be any highly transparent and physically tough polymer with a temperature/viscosity profile that will allow it to house the bichromal sphere. Once again, the fractionated polyalkylene wax of this disclosure such as the fractionated POLYWAX 1000 and/or POLYWAX 2000 may be used in the fourth material/skin.

A Gyricon display may be prepared from the microencapsulated Gyricon beads as illustrated above. Sometimes, Gyricon displays are also known as electric paper, display media, or twisted ball panel display devices, and are described, for example, in U.S. Pat. Nos. 4,126,854; 4,143,103; 4,261,653; 4,438,160; 5,389,945. In an exemplary Gyricon display, the microencapsulated Gyricon beads are sandwiched between two indium tin oxide coated substrates, such as glass or MYLAR®.

A typical process for forming the bichromal balls described herein is as follows. After extraction, the fractionated polyalkylene wax is mixed with a first pigment to produce a first wax material. The fractionated polyalkylene wax is mixed with a second pigment to produce a second wax material. These mixing operations can be performed to produce many different wax materials, typically having different colors or other different properties as compared to the other materials.

Next, the wax materials prepared are then heated to a temperature greater than the highest melting temperature of the wax materials. The heating operations can be performed separately upon each of the wax materials or collectively. Upon the wax materials being heated to a suitable temperature such that the wax material flows, the materials are then deposited onto a spinning disk to produce bichromal balls adapted for use in high temperature applications. The spinning disk production method is described in one or more of the patents referenced herein.

The polymer or wax materials can be colored through colorants such as pigments, dyes, light reflective or light blocking particles, etc., as it is commonly known in the art. A “colorant” as used herein is any substance that imparts color to another material or mixture. Colorants, such as, for example, dyes or pigments, may either be (1) naturally present in a material, (2) admixed with it mechanically, or (3) applied to it in a solution.

In this regard, a “pigment” is defined herein to include any substance, usually in the form of a dry powder, which imparts color to another substance or mixture. Most pigments are insoluble in organic solvents and water; exceptions are the natural organic pigments, such as chlorophyll, which are generally organosoluble.

Pigments may be classified as follows:

I. Inorganic
(a) metallic oxides (iron, titanium, zinc, cobalt, chromium).
(b) metal powder suspensions (gold, aluminum).
(c) earth colors (siennas, ochers, umbers).
(d) lead chromates.
(e) carbon black.

II. Organic
(a) animal (rhodopsin, melanin).
(b) vegetable (chlorophyll, xantrophyll, indigo, flavone, carotene).

Some pigments (zinc oxide, carbon black) are also reinforcing agents, but the two terms are not synonymous; in the parlance of the paint and rubber industries these distinctions are not always observed.

“Dyes” include natural and synthetic dyes. A natural dye is an organic colorant obtained from an animal or plant source. Among the best-known are madder, cochineal, logwood, and indigo. The distinction between natural dyes and natural pigments is often arbitrary.

A synthetic dye is an organic colorant derived from coal-tar- and petroleum-based intermediates and applied by a variety of methods to impart bright, permanent colors to textile fibers. Some dyes, call “fugitive,” are unstable to sunlight, heat, and acids or bases; others, called “fast,” are not. Direct (or substantive) dyes can be used effectively without “assistants”; indirect dyes require either chemical reduction (vat type) or a third substance (mordant), usually a metal salt or tannic acid, to bind the dye to the fiber.

There may be no generally accepted distinction between dyes and pigments. Some have proposed one on the basis of solubility, or of physical form and method of application. Most pigments, so called, are insoluble, inorganic powders, the coloring effect being a result of their dispersion in a solid or liquid medium. Most dyes, on the other hand, are soluble synthetic organic products which are chemically bound to and actually become part of the applied material. Organic dyes are usually brighter and more varied than pigments, but tend to be less stable to heat, sunlight, and chemical effects. The term colorant applies to black and white as well as to actual colors.

In various embodiments, conventional colorant materials may be used, such as Color Index (C.I.) Solvent Dyes, Disperse Dyes, modified Acid and Direct Dyes, Basic Dyes, Sulphur Dyes, Vat Dyes, and the like. Examples of suitable dyes include Neozopon Red 492 (BASF); Orasol Red G (Ciba-Geigy); Direct Brilliant Pink B (Crompton & Knowles); Aizen Spilon Red C-BH (Hodogaya Chemical); Kayanol Red 3BL (Nippon Kayaku); Levanol Brilliant Red 3BW (Mobay Chemical); Levaderm Lemon Yellow (Mobay Chemical); Spirit Fast Yellow 3G; Aizen Spilon Yellow C-GNH (Hodogaya Chemical); Sirius Supra Yellow GD 167; Cartasol Brilliant Yellow 4GF (Sandoz): Pergasol Yellow CGP (Ciba-Geigy); Orasol Black RLP (Ciba-Geigy); Savinyl Black RLS (Sandoz); Dermacarbon 2GT (Sandoz); Pyrazol Black BG (ICI); Morfast Black Conc. A (Morton-Thiokol): Dioazol Black RN Quad (ICI); Orasol Blue GN (Ciba-Geigy); Savinyl Blue GLS (Sandoz); Luxol Blue MBSN (Morton-Thiokol); Sevron Blue 5GMF (ICI); Basacid Blue 750 (BASF), Neozapon Black X51 [C.I. Solvent Black, C.I. 12195] (BASF), Sudan Blue 670 [C.I. 61554] (BASF), Sudan Yellow 146 [C.I. 12700] (BASF), Sudan Red 462 [C.I. 26050] (BASF), Intratherm Yellow 346 from Crompton and Knowles, C.I. Disperse Yellow 238, Neptune Red Base NB543 (BASF, C.I. Solvent Red 49), Neopen Blue FF-4012 from BASF, Lampronol Black BR from ICI (C.I. Solvent Black 35), Morton Morplas Magenta 36 (C.I. Solvent Red 172), metal phthalocyanine colorants such as those disclosed in U.S. Pat. No. 6,221,137, the disclosure of which is totally incorporated herein by reference, and the like. Polymeric dyes can also be used, such as those disclosed in, for example, U.S. Pat. Nos. 5,621,022 and 5,231,135, the disclosures of each of which are totally incorporated herein by reference, and commercially available from, for example, Milliken & Company as Milliken Ink Yellow 869, Milliken Ink Blue 92, Milliken Ink Red 357, Milliken Ink Yellow 1800, Milliken Ink Black 8915-67, uncut Reactant Orange X-38, uncut Reactant Blue X-17, and uncut Reactant Violet X-80.

Examples of suitable pigments include Violet Toner VT-8015 (Paul Uhlich); Paliogen Violet 5100 (BASF); Paliogen Violet 5890 (BASF); Permanent Violet VT 2645 (Paul Uhlich); Heliogen Green L8730 (BASF); Argyle Green XP-111-S (Paul Uhlich); Brilliant Green Toner GR 0991 (Paul Uhlich); Lithol Scarlet D3700 (BASF); Toluidine Red (Aldrich); Scarlet forThermoplast NSD PS PA (Ugine Kuhlmann of Canada): E.D. Toluidine Red (Aldrich): Lithol Rubine Toner (Paul Uhlich): Lithol Scarlet 4440 (BASF); Bon Red C (Dominion Color Company); Royal Brilliant Red RD8192 (Paul Uhlich); Oracet Pink RF (Ciba-Geigy); Paliogen Red 3871 K (BASF); Paliogen Red 3340 (BASF); Lithol Fast Scarlet L4300 (BASF); Heliogen Blue L6900, L7020 (BASF); Heliogen Blue K6902, K6910 (BASF); Heliogen Blue D6840, D7080 (BASF); Sudan Blue OS (BASF); Neopen Blue FF4012 (BASF); PV Fast Blue B2G01 (American Hoechst); Irgalite Blue BCA (Ciba-Geigy): Paliogen Blue 6470 (BASF): Sudan III (Red Orange) (Matheson, Colemen Bell); Sudan II (Orange) (Matheson, Colemen Bell); Sudan Orange G (Aldrich). Sudan Orange 220 (BASF); Paliogen Orange 3040 (BASF); Ortho Orange OR 2673 (Paul Uhlich); Paliogen Yellow 152,1560 (BASF); Lithol Fast Yellow 0991 K (BASF); Paliotol Yellow 1840 (BASF); Novoperm Yellow FGL (Hoechst); Permanent Yellow YE 0305 (Paul Uhlich); Lumogen Yellow D0790 (BASF); Suco-Yellow L1250 (BASF); Suco-Yellow D1355 (BASF); Suco Fast Yellow D1355, D1351 (BASF); Hostaperm Pink E (American Hoechst): Fanal Pink D4830 (BASF): Cinquasia Magenta (DuPont); Paliogen Black L0084 (BASF); Pigment Black K801 (BASF); and carbon blacks such as REGAL 3300 (Cabot), Carbon Black 5250, Carbon Black 5750 (Columbia Chemical), and the like. Also included are black pigments set forth above.

Other examples of suitable colorants (i.e., pigments, dyes, etc.) include, but are not limited to, magenta pigments such as 2,9-dimethyl-substituted quinacridone and anthraquinone dye, identified in the color index as C1 60710, C1 Dispersed Red 15, a diazo dye identified in the color index as C1 26050, C1 Solvent Red 19, and the like; cyan pigments including copper tetra-4-(octadecylsulfonamido) phthalocyanine, copper phthalocyanine pigment, listed in the color index as C1 74160, Pigment Blue, and Anthradanthrene Blue, identified in the color index as C1 69810, Special Blue X-2137, and the like; yellow pigments including diarylide yellow 3,3-dichlorobenzidine acetoacetanilides, a monoazo pigment identified in the color index as C1 12700, C1 Solvent Yellow 16, a nitrophenyl amine sulfonamide identified in the color index as Foron Yellow SE/GLN, C1 Dispersed Yellow 33, 2,5-dimethoxy acetoacetanilide, Permanent Yellow FGL, and the like.

Examples of black pigments include carbon black products from Cabot corporation, such as Black Pearls 2000, Black Pearls 1400, Black Pearls 1300, Black Pearls 1100, Black Pearls 1000, Black Pearls 900, Black Pearls 880, Black Pearls 800, Black Pearls 700, Black Pearls 570, Black Pearls 520, Black Pearls 490, Black Pearls 480, Black Pearls 470, Black Pearls 460, Black Pearls 450, Black Pearls 430, Black Pearls 420, Black Pearls 410, Black Pearls 280, Black Pearls 170, Black Pearls 160, Black Pearls 130, Black Pearls 120, Black Pearls L; Vulcan XC72, Vulcan PA90, Vulcan 9A32, Regal 660, Regal 400, Regal 330, Regal 350, Regal 250, Regal 991, Elftex pellets 115, Mogul L. Carbon black products from Degussa-Hüls such as FW1, Nipex 150, Printex 95, SB4, SB5, SB100, SB250, SB350, SB550; Carbon black products from Columbian such as Raven 5750; Carbon black products from Mitsubishi Chemical such as #25, #25B, #44, and MA-100-S can also be utilized.

Moreover, one or more dispersing aids, such as surface active agents and dispersants aids like Aerosol™ OT-100 (from American Cynamid Co. of Wayne, N.J.) and aluminum octoate (Witco) and OLOA 11000, OLOA 11001, OLOA 11002, OLOA 11005, OLOA 371, OLOA 375, OLOA 411, OLOA 4500, OLOA 4600, OLOA 8800, OLOA 8900, OLOA 9000, OLOA 9200 and the like (from Chevron of Houston Tex.). Dispersant aids such as X-5175 (from Baker-Petrolite Corp.), Unithox™ 480 (from Baker-Petrolite Corp.), Polyox™ N80 (Dow), and Ceramer™ 5750 (Baker-Petrolite Corp.) can further be added to the waxy base material. Other dispersing aids such as Ceridust 5551, Ceridust 32451, Ceridust 3910 from Clariant Corp. can also be included.

Once the high temperature bichromal balls are produced by the process set forth above, they may be encapsulated for use in high temperature display applications. Generally, the encapsulation process involves providing a silicone oil which as previously noted can be polydimethylsiloxane. A shell material as described in the art is also provided. The high temperature bichromal balls, i.e. those utilizing the fractionated polyalkylene wax, are then encapsulated. The bichromal balls are dispersed in the silicone oil within a shell of the shell material.

Specific embodiments of the disclosure will now be described in detail. These examples are intended to be illustrative, and the disclosure is not limited to the materials, conditions, or process parameters set forth in these embodiments. All parts and percentages are by weight unless otherwise indicated.

### EXAMPLES

### Example 1

### Fractionalization Process

150-gallon POLYWAX 2000 Extraction Process

50 kg POLYWAX 2000 (Baker-Petrolite Corp.), see Table 1, and 292 kg ISOPAR/Ashpar C (Ashland) were charged into a 150-gallon Cogeim filter-dryer that was fitted with a 0.5 um Gortex filter cloth. Mixing was started at 30 RPM, the filter-dryer was heated to 85° C., and the slurry was mixed for three hours at 85° C. The ASHPAR C was filtered off by vacuum, leaving a POLYWAX 2000 wet cake on the filter cloth. 292 kg fresh Ashpar C was charged into the filter-dryer, and the POLYWAX 2000 wet cake was reslurried by mixing at 30 RPM. The filter-dryer was again heated to 85° C., the slurry was mixed for three hours at 85° C., and the Ashpar C was filtered off by vacuum. The preceding was repeated two more times, for a total of four mixing/filtering steps. The remaining POLYWAX 2000 wet cake was dried at 85° C. for 18 hours in the filter-dryer, and then discharged as a fine white powder. The powder was comilled through a 60-mesh screen to remove lumps. The final product from this procedure will hereafter be referred to as “fractionated POLYWAX 2000”.

POLYWAX 1000 Extraction Process

20 grams of powdered POLYWAX 1000 was suspended in 160 ml of ISOPAR C. The suspension was heated up to 70° C. with magnetic stirring for one hour and settled inside a 70° C. oven for another hour. The clear supernatant solvent was decanted as much as possible. Upon cooling the solvent to room temperature, a white powder was precipitated and isolated by filtration. The isolated solid was dried in a vacuum oven at 50° C. overnight (about 14 to about 18 hours) to remove the absorbed ISOPAR C. 1.88 grams (9.4%) was recovered.

### Example 2

DSC Characterization

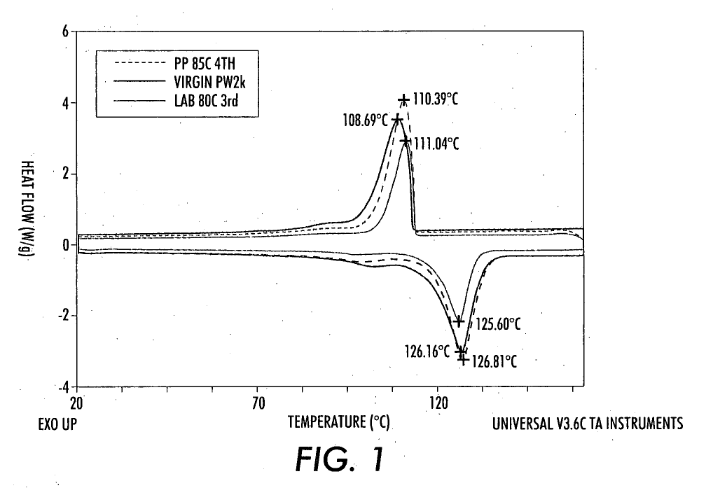

Three different samples are tested by DSC: virgin PW2000, pilot plant fractionated PW2000 and bench-scale PW2000. The DSC traces are shown below in FIG. 1. The virgin PW2000 exhibits a broad endothermic event from 90-110° C., which is higher than both fractionated samples. In addition, the pilot plant sample (pp) shows a more silent feature than the bench scale (lab) sample. Therefore, the pilot plant sample is more pure than bench scale one.

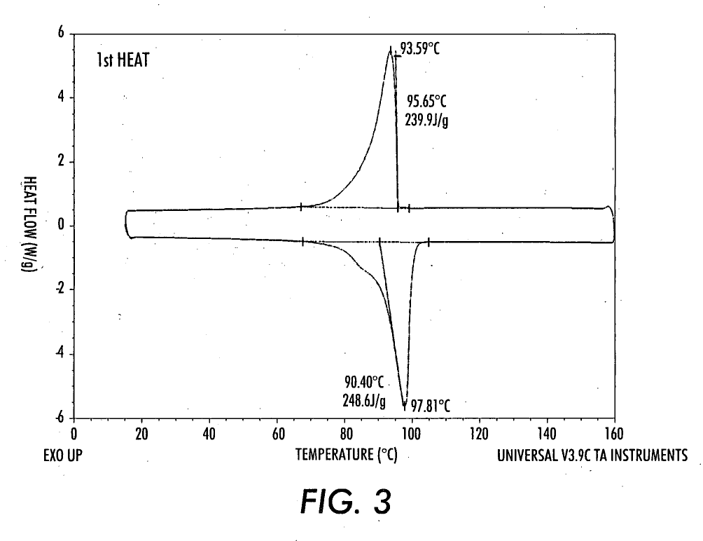

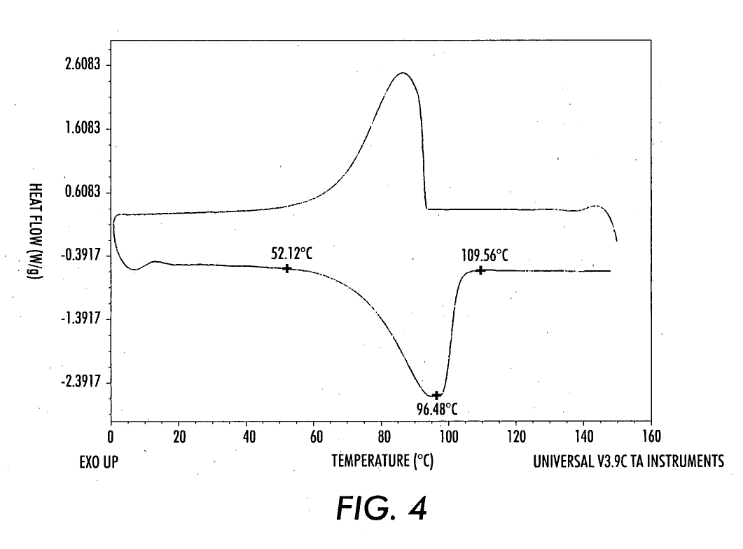

Fractionated POLYWAX 1000 and virgin POLYWAX 655 were also tested. FIGS. 3 and 4 compare the DSC traces for the first portion extracted from POLYWAX 1000 and the DSC trace for POLYWAX 655. The DSC traces reflect the Mw distribution for the polyalkylenes. As shown in FIG. 3, the melting process for the first portion extracted from POLYWAX 1000, the melting process initiates at about 65° C. and completes at about 105° C.,. The peak temperature, which reflects the melting point, is 97° C. and is very close to that of POLYWAX 655 (which is around 96° C.). The melting characteristic for the first portion, which is the difference between the temperature at which the melting process ends and the temperature at which it begins, is relatively narrow and around 40° C. POLYWAX 655, on the other hand, exhibits a broad melting characteristic of about 57° C. as evidenced by the DSC trace which shows that the melting process begins at about 52° C. and ends at about 109.5° C.

### Example 3

High Temperature GPC (HT-GPC) Results

Table 1 shows molecular weight characteristics that were measured for three wax samples using a high temperature GPC technique. Table 1 indicates that the fractionated POLYWAX 2000 has a higher molecular weight and narrower polydispersity than the unfractionated material. Also, analysis of the residue shows that low molecular weight impurities are being removed from the POLYWAX.

TABLE 1
HTGPC Analysis of POLYWAX samples

| Samples Description | Mn | Mw | PDI |
|---|---|---|---|
| Starting POLYWAX 2000 | 1890 | 2746 | 1.45 |
| | | (Mw) | |
| Fractionated POLYWAX 2000 (the 2^{nd} | 2694 | 3418 | 1.27 |
| portion) | | (Mw₂) | (PDI₂) |
| Residue removed from POLYWAX | 1557 | 1999 | 1.28 |
| 2000 by extraction process (the 1^{st} | | (Mw₁) | (PDI₁) |
| portion) | | | |
| Starting POLYWAX 1000 | 1154 | 1243 | 1.08 |
| | | (Mw) | |
| Fractionated POLYWAX 1000 (the 2^{nd} | 1259 | 1325 | 1.05 |
| portion) | | (Mw₂) | (PDI₂) |
| Residue removed from POLYWAX | 840 | 872 | 1.04 |
| 1000 by extraction process (the 1^{st} | | (Mw₁) | (PDI₁) |
| portion), PW 1000-F1 | | | |

### Example 4

HT-GPC Statistical Analysis

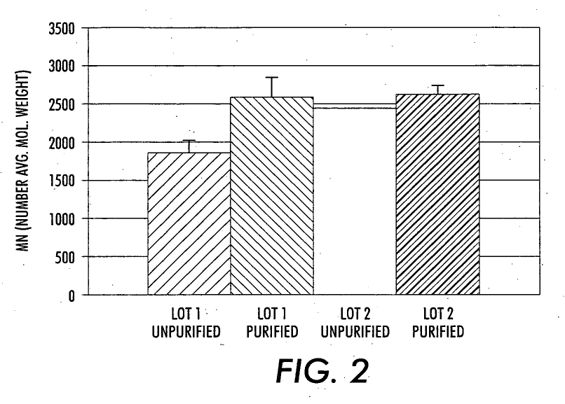

FIG. 2 shows HT-GPC statistical analysis of several fractionated and unfractionated POLYWAX samples (95% confidence interval is indicated by error bars). The figure indicates that the fractionated material indeed has a consistently higher number average molecular weight than the unfractionated material. Also, the two different lots of unfractionated POLYWAX have significantly different Mn. The fractionalization process thus creates a more consistent supply of wax for processing into the final application.

### Example 5

Electrical Analysis

This example demonstrates the advantage of Fractionated PW2000 over regular PW2000. Three Gyricon samples made of two different polywax were tested side by side: Starting PW2000 and Fractionated PW2000. The Contrast Ratio, CR of Starting PW2000 dropped after 48 hours, and Fractionated PW2000 sustained its CR. See Table 2 below.

TABLE 2
Electrical analysis of Gyricon devices
made using starting and fractionated POLYWAX

| | | | |
|---|---|---|---|
| 60 V | 80 V | 100 V | 125 V |

Starting PW2000

| | | | | |
|---|---|---|---|---|
| AA531, XRCC531 | | | | |
| Time zero | 2.13 | 3.45 | 4.31 | 4.49 |
| 48 hours | 1.16 | 1.34 | 1.55 | 1.86 |

Fractionated PW2000

| | | | | |
|---|---|---|---|---|
| AA569, XRCC94 | | | | |
| Time zero | 3.67 | 3.91 | 3.76 | 3.57 |
| 48 hours | 3.55 | 3.64 | 3.56 | 3.40 |
| 120 hours | 3.26 | 3.60 | 3.60 | 3.50 |

Gyricon devices were shown to have increased contrast ratio value when using the fractionated materials of this disclosure.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

1. A bichromal bead comprising a polyalkylene fraction obtained by (i) combining an initial polyalkylene having a weight average molecular weight Mw with a fluid, wherein the fluid is selected from the group consisting of a) acyclic branched or unbranched hydrocarbons having the general formula CₙH₂ₙ₊₂, in which n is the number of atoms and is from about 1 to about 30; and, b) supercritical fluids, (ii) dissolving a first portion of the polyalkylene in the fluid, the first portion having a weight average molecular weight Mw₁ which is less than Mw, (iii) separating a second portion of the polyalkylene, the second portion being suitably insoluble in the fluid, the first portion having a weight average molecular weight Mw₂ which is greater than Mw, and (iv) recovering the first portion and the second portion of the polyalkylene,
wherein the polyalkylene fraction is selected from at least one of the first portion or the second portion of the polyalkylene.

2. The bichromal bead according to claim 1, wherein the polyalkylene fraction comprises the first portion of the polyalkylene.

3. The bichromal bead according to claim 1, wherein the polyalkylene fraction comprises the second portion of the polyalkylene.

4. The bichromal bead according to claim 1, wherein the first portion of the polyalkylene has a weight average molecular weight Mw₁ that is from about 0.55 Mw to about 0.95 Mw and the second portion of the polyalkylene has a weight average molecular weight Mw₂ that is from about 1.05 Mw to about 1.45 Mw.

5. The bichromal bead according to claim 1, wherein the first portion of the polyalkylene has a polydispersity of from approaching 1 to about 1.30.

6. The bichromal bead according to claim 1, wherein the first portion of the polyalkylene has a polydispersity of approaching 1 to about 1.07.

7. The bichromal bead according to claim 1, wherein the first portion of the polyalkylene has a polydispersity of approaching 1 to about 1.05.

8. The bichromal bead according to claim 1, wherein the first portion of the polyalkylene exhibits a melting characteristic of about 40° C. or less.

9. The bichromal bead according to claim 1, wherein the first portion of the polyalkylene exhibits a crystallization characteristic of about 50° C. or less.

10. The bichromal bead according to claim 1, wherein n is from about 4 to about 26.

11. The bichromal bead according to claim 1, wherein n is from about 5 to about 16.

12. The bichromal bead according to claim 1, wherein the end temperature of the crystallization process, T₄ of the polyalkylene fraction is from about 1° C. to about 20° C. above the temperature T₄ of the initial polyalkylene.

13. A bichromal bead comprising:
a colorant; and
a polyalkylene wax fraction obtained by extracting the polyalkylene wax fraction from a starting polyalkylene with an acyclic branched or unbranched hydrocarbons having the general formula CₙH₂ₙ₊₂, in which n is the number of atoms and is from about 1 to about 30 and recovering the polyalkylene wax fraction, the polyalkylene wax fraction having a polydispersity of approaching 1 to about 1.30.

14. The bichromal bead according to claim 13, wherein the first portion of the polyalkylene wax fraction has a polydispersity of approaching 1 to about 1.07.

15. The bichromal bead according to claim 13, wherein the first portion of the polyalkylene wax fraction has a polydispersity of approaching 1 to about 1.05.

16. The bichromal bead according to claim 13, wherein the polyalkylene wax fraction comprises hydrocarbon fractions of from about 0 to about 50 carbons.

17. The bichromal bead according to claim 13, wherein the polyalkylene wax fraction exhibits a melting transition having a range of about 50° C. or less as determined by DSC.

18. A bichromal bead comprising:
a colorant; and
a carrier comprising a first polyalkylene wax portion separated from a starting polyalkylene wax having a polydispersity index PDI, the polyalkylene wax portion being separated from the starting polyalkylene wax by (i) combining the starting polyalkylene wax with a fluid comprising acyclic branched or unbranched hydrocarbons having the general formula CₙH₂ₙ₊₂, in which n is the number of atoms and is from about 1 to about 30, (ii) dissolving the first polyalkylene wax portion in the fluid, and (iii) recovering the first polyalkylene wax portion from the fluid, wherein the first polyalkylene wax portion has a polydispersity index PDI₁, and PDI₁ is less than PDI.

19. The bichromal bead according to claim 18, wherein mixing the starting polyalkylene wax with the fluid and dissolving the first polyalkylene wax portion are carried out a temperature of from about 60° C. to about 90° C.

20. The bichromal bead according to claim 18, wherein the first polyalkylene wax portion has a melting characteristic of about 40° C. or less.

21. The bichromal bead according to claim 18, wherein the mixing of the starting polyalkylene wax with the fluid and dissolving the first polyalkylene wax portion are carried at a temperature of about 70° C.
